(19) United States
(12) Patent Application Publication
(10) Pub. No.: US 2012/0077714 A1
(43) Pub. Date: Mar. 29, 2012

(54) Mass Spectrometry Based Particle Separation

(76) Inventors: Garry P. Nolan, San Francisco, CA (US); Sean C. Bendall, San Mateo, CA (US); Peter O. Krutzik, Los Altos, CA (US)

(21) Appl. No.: 13/236,070

(22) Filed: Sep. 19, 2011

Related U.S. Application Data
(60) Provisional application No. 61/384,460, filed on Sep. 20, 2010.

(51) Int. Cl. H01J 49/26; G01N 33/00; C40B 40/10; C07K 16/00; C12M 1/34

(52) U.S. Cl. 506/18; 435/7.2; 436/501; 530/391.3; 435/287.2; 422/68.1; 250/288

(57) ABSTRACT

Certain embodiments provide a method of sample analysis that comprises: a) labeling a particle using a specific binding reagent that is cleavably linked to an elemental tag; b) flowing the labeled particle through a flow cell of a mass cytometer; c) cleaving the elemental tag from the labeled particle; d) performing elemental analysis of the cleaved elemental tag without destroying the particle, to produce data; e) matching data for the particle with the particle; and f) collecting the particle. Also provided is a specific binding reagent that is cleavably linked to an elemental tag, and a mass cytometer adapted to perform the method.

### CROSS-REFERENCE TO RELATED APPLICATION

This application claims the benefit of priority to U.S. Provisional Patent Application No. 61/384,460 filed on Sep. 20, 2010, which is hereby expressly incorporated by reference in its entirety.

### GOVERNMENT RIGHTS

This invention was made with Government support under contracts HV028183 and CA130826 awarded by the National Institutes of Health. The Government has certain rights in this invention.

### BACKGROUND

In traditional flow cytometry, fluorescently labeled particles (e.g., live cells, fixed cells or beads, etc) are individually distinguished and separated based on their fluorescence and light scatter characteristics. The phenotype of the particles can be further investigated after they are isolated. Such traditional flow cytometry methods are limited by the number of simultaneous parameters that can be measured on a single particle.

Elemental mass spectrometry-based flow cytometry (also known as “mass cytometry”) offers a new approach to analyze cells by replacing the fluorochrome-labed binding reagents (e.g., the fluorescently labeled antibodies) with binding reagents that are “mass tagged”, i.e., tagged with an element or isotope having a defined mass. In these methods, the labeled particles are introduced into a mass cytometer, where they are individually atomized and ionized. The individual particles are then subjected to elemental analysis, which identifies and measures the abundance of the mass tags used. The identities and the amounts of the isotopic elements associated with each particle are then stored and analyzed. Due to the resolution of elemental analysis and the number of elemental isotopes that can be used, it is possible to simultaneously measure up to 100 or more parameters on a single particle by without experiencing spectral overlap. The value of mass cytometry methods is limited, however, because the particles are destroyed during their analysis.

### SUMMARY

Provided herein is a method of sample analysis that comprises: a) labeling a particle using a specific binding reagent that is cleavably linked to an elemental tag; b) passing the labeled particle through a flow cell of a mass cytometer; c) cleaving the elemental tag from the labeled particle in a controlled fashion; d) performing elemental analysis of the cleaved elemental tag without destroying the particle, to produce data; e) matching data for the particle with the particle; and f) collecting the particle. Also provided is a specific binding reagent that is cleavably linked to an elemental tag, and a mass cytometer adapted to perform the method.

### BRIEF DESCRIPTION OF THE FIGURES

FIG. 1 schematically illustrates an exemplary embodiment of the method described in greater detail below.

### DEFINITIONS

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

As used herein, the term “labeling” refers to attaching a detectable moiety to an analyte such that the presence and/or abundance of the analyte can be determined by evaluating the presence and/or abundance of the label.

As used herein, the term “multiplexing” refers to using more than one label for the simultaneous or sequential detection and measurement of biologically active material.

As used herein, the term “particle” refers to a three dimensional object in the range of 100 nm to 1 mm , e.g., 1 μm to 100 μm, in size. Single cells (which may be living or fixed) and polymer beads are examples of particles.

As used herein, the term “labeled specific binding reagent” refers to a labeled reagent that can specifically bind to binding sites on the surface of a particle. Such labeled specific binding reagents contain a specific binding reagent, e.g., an antibody or an aptamer, and a label that may or may not be covalently bound to the specific binding reagent.

As used herein, the terms “antibody” and “immunoglobulin” are used interchangeably herein and are well understood by those in the field. Those terms refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, a fluorescent molecule, or a stable elemental isotope and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986),).

An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Chimeric antibodies are antibodies whose light and heavy chain genes have been constructed, typically by genetic engineering, from antibody variable and constant region genes belonging to different species. For example, the variable segments of the genes from a rabbit monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody (e.g., the anti-Tac chimeric antibody made by the cells of A.T.C.C. deposit Accession No. CRL 9688), although other mammalian species may be used.

The term “specific binding” refers to the ability of a binding agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold).

In certain embodiments, the affinity between a binding agent and analyte when they are specifically bound in a capture agent/analyte complex is characterized by a K_{D} (dissociation constant) of less than 10⁻⁶M, less than 10⁻⁷ M, less than 10⁻⁸ M, less than 10⁻⁹ M, less than 10⁻⁹ M, less than 10⁻¹¹ M, or less than about 10⁻¹² M or less.

As used herein the term “isolated”, refers to an reagent of interest that is at least 60% free, at least 75% free, at least 90% free, at least 95% free, at least 98% free, and even at least 99% free from other components with which the reagent is associated with prior to purification.

As used herein, the term “cleavably linked to” refers to a linkage that is selectively breakable using a stimulus (e.g., a physical, chemical or enzymatic stimulus) that leaves the moieties to which the linkages joins intact.

A “linkage” may be non-covalent or covalent.

As used herein, the term “mass tagged” refers to a molecule that is tagged with an element that is identifiable by its mass. Examples of elements that are identifiable by their mass include noble metals and lanthanide, although other elements may be employed. An element may exist as one or more isotopes, and this term also includes isotopes of positively and negatively metals. The terms “mass tagged” and “elementally tagged: may be used interchangeably herein.

As used herein, the term “elemental tag” means any element, including a noble metal or lanthanide, that is identifiable by its mass and used to tag a biologically active material or analyte. An elemental tag has an atomic mass that is distinguishable from the atomic masses present in the analytical sample and in the particle of interest. Elements suitable for this purpose include, but are not limited to, lanthanides and noble metals. In certain cases, an elemental tag may have an atomic number of 21-90. In particular embodiments, the elemental tag may contain a transition metal, i.e., an element having the following atomic numbers, 21-29, 39-47, 57-79, and 89. Transition elements include the lanthanides and noble metals. See, e.g., Cotton and Wilkinson, 1972, pages 528-530. The elemental tags employed herein are non-biological in that they are man made and not present in typical biological samples, e.g., cultured cells, unless they are provided exogenously.

As used herein, the term “lanthanide” means any element having atomic numbers 58 to 71. Lanthanides are also called “rare earth metals”.

As used herein, the term “noble metal” means any of several metallic elements, the electrochemical potential of which is much more positive than the potential of the standard hydrogen electrode, therefore, an element that resists oxidation. Examples include palladium, silver, iridium, platinum and gold.

As used herein, the term “inductively coupled plasma” (ICP) means a source of atomization and ionization in which a plasma is established in an inert gas (usually argon) by the inductive coupling of radiofrequency energy. The frequency of excitation force is in the MHz range.

As used herein, the term “plasma source” means a source of atoms or atomic ions comprising a hot gas (usually argon) in which there are approximately equal numbers of electrons and ions, and in which the Debye length is small relative to the dimensions of the source.

As used herein, the term “flow cell” refers to a conduit in which particles flow, in a medium, one by one in single file.

As used herein, the term “a diverter” refers to a branch of a flow cell in which particles can be separated from other components passing through the flow cell.

As used herein, the term “elemental analysis” refers to a method by which the presence and/or abundance of elements of a sample are evaluated.

As used herein, the term “mass cytometry” refers to a method in which particles are separated from one another by use of a flow cell and then subjected to elemental analysis.

As used herein, the term “destroying” refers to breaking apart, e.g., into atoms, i.e., atomizing.

As used herein, the term “matching”, in the context of matching one thing to another, refers to providing an association (e.g., a linkage that may be direct or indirect, e.g., via a key) that links those things together. When data is matched to a sample from which the data was obtained, the matching may be referred to as “registering”.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

A used herein, an “aptamer” is a synthetic oligonucleotide or peptide molecule that specifically binds to a specific target molecule.

Other definitions of terms may appear throughout the specification.

### DETAILED DESCRIPTION OF EXEMPLARY EMBODIMENTS

In order to further illustrate the present invention, the following specific examples are given with the understanding that they are being offered to illustrate the present invention and should not be construed in any way as limiting its scope.

### Method for Sample Analysis

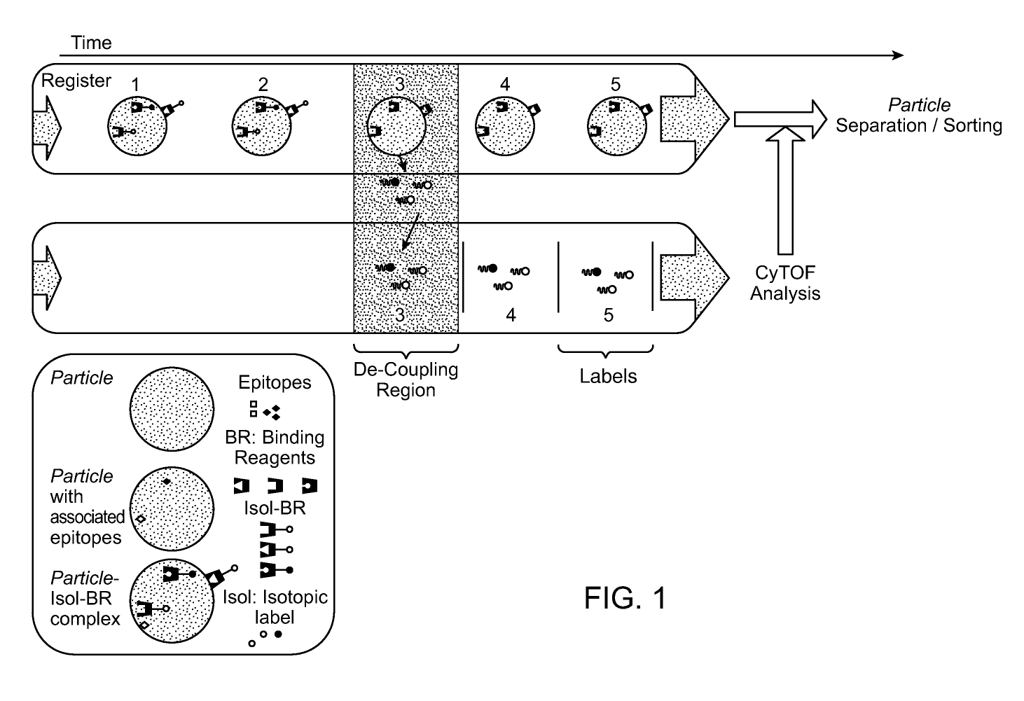

Provided herein is a method of sample analysis. In general terms, a suspension of single particles is labeled with a labeled specific binding reagent that contains a cleavably linked elemental tag to produce a labeled particle. The labeled particle is introduced into the flow cell of a mass cytometer where it is placed in single file with other labeled particles. The elemental tag is then cleaved from the labeled particle, either in the flow cell or after the particle has exited the flow cell. In one embodiment, the cleavage stimulus may be applied to the labeled particle as the labeled particle is passing through the flow cell. After the elemental tag has been cleaved from the particle, the cleaved tag and the particle are separated, and the cleaved tag is subject to elemental analysis (by, e.g., ICP-MS), without destroying the particle, to produce data. In certain cases, the data obtained for the particle may be matched with the particle (e.g., associated with the physical location of the particle), and the particle may be collected. In certain cases, a particle may be separated from other particles based on results obtained for that particle. An exemplary embodiment of this method is illustrated in FIG. 1.

In one embodiment, the method may be performed on a single cell suspension or beads for example. If cells are employed, the single cell suspension may contain cells from a microorganism, e.g., a pathogen (e.g., bacteria), disaggregated cells grown in culture, blood cells, or disaggregated cells of a tissue, for example. Such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known in the art. Cells may be obtained from fixed or unfixed, fresh or frozen, whole or disaggregated samples. Disaggregation of tissue may occur either mechanically or enzymatically. In particular embodiments, the cells may be obtained from a blood draw, from lymphoid tissue, e.g., spleen, lymph nodes, thymus, bone marrow or cultured cells. Cells from solid tissues may be mechanically treated or treated with an enzyme to produce a single cell suspension using known techniques. If beads are employed, the beads can range in size from 20 nM to 200 μM or larger, and may be made of polystyrene, but other materials such as polymethylmethacrylate (PMMA), polyvinyltoluene (PVT), styrene/butadiene (S/B) copolymer, styrene/vinyltoluene (S/VT) can also used. Reactive groups commonly used include carboxyl, amino, aldehyde, hydroxyl, epoxy, and chloromethyl (See, e.g., U.S. Pat. Nos. 4,217,338, 5,326,692, 5,786,219, 4,717,655, 7,445,8445,573,909 and 6,023,540). To these reactive groups other types of linkers can be attached. Beads as described above can be obtained commercially from numerous sources including Molecular Probes (Invitrogen), Bangs Labs, and Polymicrospheres, Inc.

The particles are then labeled using a specific binding reagent, e.g., an antibody, that comprises a cleavably linked elemental tag. Methods for labeling particles with binding reagents are known. In particular embodiments, the cells are labeled with a reagent that binds to a cell surface marker, e.g., GD2, EGF-R, CEA, CD52, CD20, Lym-1, CD6, complement activating receptor (CAR), EGP40, VEGF, tumor-associated glycoprotein TAG-72 AFP (alpha-fetoprotein), BLyS (TNF and APOL—related ligand), CAl25 (carcinoma antigen 125), CEA (carcinoembrionic antigen), CD2 (T-cell surface antigen), CD3 (heteromultimer associated with the TCR), CD4, CD11a (integrin alpha-L), CD14 (monocyte differentiation antigen), CD20, CD22 (B-cell receptor), CD23 (low affinity IgE receptor), CD25 (IL-2 receptor alpha chain), CD30 (cytokine receptor), CD33 (myeloid cell surface antigen), CD40 (tumor necrosis factor receptor), CD44v6 (mediates adhesion of leukocytes), CD52 (CAMPATH-1), CD80 (costimulator for CD28 and CTLA-4), complement component C5, CTLA, EGFR, eotaxin (cytokine A11), HER2/neu, HER3, HLA-DR, HLA-DR10, HLA ClassII, IgE, GPiib/iiia (integrin), Integrin aVβ3, Integrins a4β1 and a4β7, Integrin β2, IFN-gamma, IL-1β, IL-4, IL-5, IL-6R (IL6 receptor), IL-12, IL-15, KDR (VEGFR-2), lewisy, mesothelin, MUC1, MUC18, NCAM (neural cell adhesion molecule), oncofetal fibronectin, PDGFβR (Beta platelet-derived growth factor receptor), PMSA, renal carcinoma antigen G250, RSV, and E-Selectin, etc.

Cells are introduced into a fluidic system and hydrodynamically focused one cell at a time through a flow cell using a sheath fluid . In particular embodiments, the particle may be compartmentalized in the flow cell by introduction of an immiscible barrier, e.g., using a gas (e.g., air or nitrogen) or oil, such that the particle is physically separated from other particles that are passing through the flow cell. The particles may be compartmentalized prior to or during introduction of the particle into the flow cell by introducing an immiscible material (e.g., air or oil) into the flow path. Compartmentalization of the particle is done before the elemental tag is cleaved from the particle.

After compartmentalization, the elemental tag may be cleaved from the particle in the compartment such that the particle and the free elemental tag cleaved from that particle are separated from other particles and their cleaved tags that are passing through the flow cell. After the mass tag is cleaved from the particle, the particle is separated from the cleaved mass tag by any one of a variety of different physical means, using, e.g., water pressure, electrical charge or using magnetism, methods for which are known and may be adapted from the cell sorting arts. In one embodiment, centrifugal force may be employed. In this embodiment, the compartment containing the particle and cleaved mass tag may be flowed around a corner in the flow cell, and the particle becomes separated from the cleaved mass tag in the compartment because the particle selectively travels around the outside of the corner by centrifugal force. The use of a diverter, i.e., a line splitter, allows the particles to be split off into a separate flow path from the cleaved mass tags. In another embodiment, the particles could be separately coupled to magnetically active nanoparticles which are not cleavable, and a stable magnet (with or without additional centrifugal force) could be used separate the particle from the cleaved product down the diverter. In this case, the particles are attracted to the magnet while the mass tags proceed down the path of the sheath fluid, largely unaffected by the magnet. In another embodiment the particles could be acoustically focused or diverted is away from the cleaved product using acoustic (sound) focusing. In this case, the particles are moved out of the stream of mass tags by their acoustic properties. Finally, particles may be separated from the mass tags by electrical properties. In this case, an electrical field is established within the flow cell near to the site that the mass tags are cleaved from the particle. When entering the electrical field, the cells and elemental tags are differentially diverted into two separate streams. In many of these embodiments the separation of the particle from the mass tag is done based on their different physical properties, e.g., magnetism, electrical conductance, inertia, acoustic reception, etc.

Once separated from the particles, the cleaved mass tag is subjected to elemental analysis using, for example, inductively coupled plasma mass spectrometry (ICP-MS) identity and/or determine the abundance of the mass tag, methods for performance of which are readily adapted from known methods (see, e.g., the references cited below). In particular embodiments, after being separated from the particle, the cleaved mass tags are vaporized, atomized and ionized by plasma (e.g., inductively coupled plasma) to produce ions that are subsequently analyzed by a mass spectrometer or emision spectroscopy to provide the identity and/or determine the abundance of the mass tag. The data produced by the elemental analysis of the mass tag is then registered with the location of the particle (which was separated from the mass tag prior to vaporization of the mass tag) from which the mass tag was cleaved, and the intact particle is collected. In particular embodiments, the data for a particle is used to separate the particle from other particles that have passed through the flow cell, thereby facilitating the collection of only particles having a particular phenotype. The separation of particles from one another may be done using methods that are used known in flow cytometry. In one exemplary embodiment, individual droplets produced from the fluid sheath using, e.g., a tunable transducer, the droplets are charged, and droplets containing a particle of interest are deflected from other droplets using deflection plates. The separated particle may be then collected.

The general principles of flow cytometry, including methods by which single cell suspensions can be made, methods by which cells can be labeled using, e.g., fluorescently labeled antibodies, methods by which cells can be separated from one another, as well as hardware that can be employed in flow cytometry, including flow cells, reagents, and computer is control systems are known and are reviewed in a variety of publications, including, but not limited to: Craig et al (Clin Lab Med. 2007 27:487-512), Ebo (Allergy. 2006 61:1028-39), Rieseberg (Appl. Microbiol. Biotechnol. 2001 56:350-60), Brown et al (Clin Chem. 2000 46:1221-9), Horsburgh et al (Transpl Immunol. 2000 8:3-15), Jonker et al (Histochem J. 1997 29: 347-64); Corberand et al (Hematol. Cell Ther. 1996 38:487-94); Othmer (Eur. J. Pediatr. 1992 151:398-406); Willman et al (Semin. Diagn. Pathol. 1989 6:3-12) and Sugarbaker et al (Int. Adv. Surg. Oncol. 1979 2:125-53), as well as U.S. Pat. Nos. 7,709,821, 7,634,126, 7,580,120, 7,561,267, 7,468,789 , 7,369,231, 7,300,763 , 7,299,135 , 7,113,266, 7,092,078, 7,024,316, 6,867,899, 6,861,265, and 6,813,017, for example, which publications are incorporated by reference herein for disclosure of those methods and hardware.

Likewise, the general principles of mass cytometry, including methods by which single cell suspensions can be made, methods by which cells can be labeled using, e.g., mass-tagged antibodies, methods for atomizing particles and methods for performing elemental analysis on particles, as well as hardware that can be employed in mass cytometry, including flow cells, ionization chambers, reagents, mass spectrometers and computer control systems are known and are reviewed in a variety of publications including, but not limited to Bandura et al Analytical Chemistry 2009 81 6813-6822), Tanner et al (Pure Appl. Chem 2008 80: 2627-2641), U.S. Pat. No. 7,479,630 (Method and apparatus for flow cytometry linked with elemental analysis) and U.S. Pat. No. 7,135,296 (Elemental analysis of tagged biologically active materials); and published U.S. patent application 20080046194, for example, which publications are incorporated by reference herein for disclosure of those methods and hardware.

In particular embodiments, the method described above may be employed in a multiplex assay in which a heterogeneous population of cells is labeled with a plurality of distinguishably mass tagged binding agents (e.g., a number of different antibodies). As there are more than 80 naturally occurring elements having more than 250 stable isotopes, the population of cells may be labeled using at least 5, at least 10, at least 20, at least 30, at least 50, or at least 100, up to 150 or more different binding agents (that bind to, for example different cell surface markers) that are each tagged with a different mass. After the population of cells is labeled, the cells are introduced into the flow cell, individually analyzed using the is method described above, and the cells are separated based on the mass tags associated with each of the cells. In particular embodiments, a cell having a particular profile of mass tags is desired, and the machine performing the method may be programmed to sort cells having the profile away from other cells that do not have the profile. Such sorting methods may be adapted from those currently employed in cell sorting (e.g., FACS).

### Cleavable Mass Tags

In particular embodiments, the elemental tag that is to be linked to the binding reagent may be of the formula: R-Lc-MT, where R is a reactive group that can form a linkage with a reactive group on a specific binding reagent, Lc is a cleavable linker and MT is an elemental tag. The compound may also contain a spacer. In particular embodiments, R may be, e.g., a maleimide or halogen-containing group that is sulhydryl reactive, an N-hydroxysuccinimide (NHS)-carbonate that is amine-reactive or an N,N-diisopropyl-2-cyanoethyl phosphoramidite that is hydroxyl-reactive. Such groups react with other groups on the specific binding reagent, e.g., a cysteine or other residue of an antibody). In particular embodiments, MT may be a polymer of, e.g., 10-500 units, where each unit of the polymer contains a coordinated transition metal. Suitable reactive groups and polymers containing coordinating groups, including DOTA and DTPA-based polychetants, are described in a variety of publications, including: Manabe et al. (Biochemica et Biophysica Acta 883: 460-467 (1986)) who describes attaching up to 105 DTPA residues onto a poly-L-lysine backbone using the cyclic anhydride method and also attaching polylysine-poly-DTPA polychelants onto monoclonal antibody (anti-HLA IgG₁) using a 2-pyridyl disulphide linker achieving a substitution of up to about 42.5 chelants (DTPA residues) per site-specific macromolecule; Torchilin (U.S. Pat. No. 6,203,775) who describes a generic method for labeling antibodies that includes an antibody-reactive, lanthanide chelating compound of a generic formula; Sieving (U.S. Pat. No. 5,364,614), the abstract for describes a DOTA-based polychetant containing a polylysine backbone that is linked to a protein. Further descriptions of such moieties are described in, for example: U.S. 20080003616 (Polymer backbone element tags), U.S. Pat. No. 6,203,775 (Chelating polymers for labeling of proteins), U.S. Pat. No. 7,267,994 (Element-coded affinity tags) U.S. Pat. No. 6,274,713 (Polychelants) and U.S. Pat. No. 5,364,613 (Polychelants containing macrocyclic chelant moieties), as well as many others. is These publications are incorporated by references for their generic and specific teachings of reactive groups and polymers containing coordinating groups, as well as the methods by which such compounds can be made. In addition to the methods described in the references cited above, methods for making polymer-based elemental tags are also described in detail in Zhang et al (Agnew Chem. Int. Ed. Engl. 2007 46: 6111-6114). In addition, any chelator able to bind to metal tags can be used. These include EDTA, EGTA, and Heme. These chelators are able to bind to +1, +2, +3 ions of metal tags.

The cleavable linkers that may be employed in a subject compound include linkers that are cleavable by a physical, chemical or enzymatic stimulus, including electrophilically cleavable linkers, nucleophilic ally cleavable linkers, photocleavable linkers, metal cleavable linkers, electrolytically-cleavable, and linkers that are cleavable under reductive and oxidative conditions. Such linkers are described in great detail by Guillier et al (Chem. Rev. 2000 1000:2091-2157), which disclosure is incorporated by reference in its entirety.

Suitable cleavable sites include, but are not limited to, the following: base-cleavable sites such as esters, particularly succinates (cleavable by, for example, ammonia or trimethylamine), quaternary ammonium salts (cleavable by, for example, diisopropylamine) and urethanes (cleavable by aqueous sodium hydroxide); acid-cleavable sites such as benzyl alcohol derivatives (cleavable using trifluoroacetic acid), teicoplanin aglycone (cleavable by trifluoroacetic acid followed by base), acetals and thioacetals (also cleavable by trifluoroacetic acid), thioethers (cleavable, for example, by HF or cresol) and sulfonyls (cleavable by trifluoromethane sulfonic acid, trifluoroacetic acid, thioanisole, or the like); nucleophile-cleavable sites such as phthalamide (cleavable by substituted hydrazines), esters (cleavable by, for example, aluminum trichloride); and Weinreb amide (cleavable by lithium aluminum hydride); and other types of chemically cleavable sites, including phosphorothioate (cleavable by silver or mercuric ions) and diisopropyldialkoxysilyl (cleavable by fluoride ions). Other cleavable sites will be apparent to those skilled in the art or are described in the pertinent literature and texts (e.g., Brown (1997) Contemporary Organic Synthesis 4(3); 216-237).

In particular embodiments, a photocleavable linker (e.g., a uv-cleavable linker) may be employed. Suitable photocleavable linkers for use in a subject sensor include ortho-nitrobenzyl-based linkers, phenacyl linkers, alkoxybenzoin linkers, chromium arene complex linkers, NpSSMpact linkers and pivaloylglycol linkers, as described in Guillier et al, supra.

Exemplary linking agents that may be employed in the subject methods are described in Guillier et al, supra and Olejnik et al (Methods in Enzymology 1998 291:135-154), and further described in U.S. Pat. No. 6,027,890; Olejnik et al (Proc. Natl. Acad Sci, 92:7590-94); Ogata et al. (Anal. Chem. 2002 74:4702-4708); Bai et al (Nucl. Acids Res. 2004 32:535-541); Zhao et al (Anal. Chem. 2002 74:4259-4268); and Sanford et al (Chem Mater. 1998 10:1510-20), and are purchasable from Ambergen (Boston, Mass.; NHS-PC-LC-Biotin), Link Technologies (Bellshill, Scotland), Fisher Scientific (Pittsburgh, Pa.; PIERCE EZ-LINK™ NHS-PC-LC-Biotin) and Calbiochem-Novabiochem Corp. (La Jolla, Calif.).

In an alternative embodiment, a photoacid generator (PAG) material may be employed as a cleavage agent. Such PAGs are known in the art (see, e.g., the world wide website of Sigma-Aldrich) and include N-hydroxyphthalimide trifluoromethanesulfonate, 2-Naphthyl diphenylsulfonium triflate, bis(4-tert-butylphenyl)iodonium perfluoro-l-butanesulfonate, bis(4-tert-butylphenyl)iodoniump-toluenesulfonate, bis(4-tert-butylphenyl)iodonium trifluoromethanesulfonate, (4-Bromophenyl)diphenylsulfonium trifluoromethanesulfonate, (tert-Butoxycarbonylmethoxynaphthyl)-diphenylsulfonium triflate, and many others. In this embodiment, a PAG may be present in the flowstream (e.g., mixed with sample). When UV light is directed at an area (i.e., a localized region) of this flow stream, protons are generated within the lighted area and the pH of the area drops. Any pH-sensitive material present in the area (e.g., sensitive esters or pH-sensitive binding) would be subject to modification (i.e., cleavage, hydrolysis, binding-disruption). For example, photo-induced acid generation produced by PAG is sufficient to cleave an ester bond to release the mass tag.

In a further embodiment, the compound may contain a electrolytically-cleavable linker. In this case, the mass tag can be released via electrolytic means. Acid-cleavable linkers may also be cleaved by a change in pH. Guidance for performing such method are readily adapted from Donner et al (Biochemica 4, 2003, a publication of Roche Applied Science, Indianapolis, IN).

In particular embodiments, the linker may be an enzymatically-cleavable linker. In one exemplary embodiment, the linker may contain a polynucleotide region that can be cleaved using a nuclease, for example. The nuclease can be added to the flow cell. In another case, the linker can be a polypeptide chain that is specifically cleaved by an enzyme that is added to the flow cell. The linker can be any nucleotide (including DNA, RNA, and synthetic nucleotides) or polypeptide (including synthetic amino acid analogs) that is specifically targeted by an enzyme for cleavage.

A labeled specific binding reagent is also provided. In this embodiment, the reagent may contain a specific binding reagent (e.g., an antibody or aptamer) that specifically binds an analyte; an elemental tag, as discussed above; and a cleavable linker that joins the specific binding reagent to the elemental tag. Methods for reacting compounds that contain sulfhydryl-reactive maleimide or halogen-containing group, amine-reactive NHS carbonate groups and hydroxyl-reactive N,N-diisopropyl-2-cyanoethyl phosphoramidite groups (as well as other reactive groups) are known.

### Mass Cytometers

Also provided is a mass cytometer adapted to perform the subject method. In general terms, the mass cytometer comprises: a) a flow cell comprising: i. an input for injecting labeled particles that are labeled with a specific binding reagents each comprising a binding region that is cleavably linked to an elemental tag into the flow cell in single file; ii. means for administering a cleavage stimulus to the labeled particles to cleave the elemental tag from the labeled particle as they pass through the flow cell to produce cleaved metal tags and unlabeled particles; iii. a diverter for separating the cleaved metal tags from the unlabeled particles prior to exit of the cleaved metal tags from the flow cell; b) an inductively coupled plasma mass spectrometry system operably connected to the exit of the flow cell for elemental analysis of the cleaved metal tags to produce data. The mass cytometer may contain a register for matching data for each of the cells with a cell, thereby providing a way of collecting cells having a particular mass tag profile after they have exited the flow cell. The means for administering may provide a chemical, physical or enzymatic stimulus that cleaves the elemental tag from the labeled particles. In addition, a mass cytometer may in certain cases have a optical system for detecting optical properties of the cells, e.g, forward light scatter, side light scatter, and fluorescence. The cells can be detected using this system, and other parameters of the cells, e.g., cell size, etc., may be measured.

Except for the part of the flow cell that permits separation of the cleaved mass tags from the particles, many components of a subject system are known may be adapted from cell sorters and mass cytometry systems that are known in the art. Exemplary cell sorters are described in references cited above as well as published patent application 20100105074, and mass cytometry machines may be adapted from the following: U.S. Pat. No. 7,479,630 (Method and apparatus for flow cytometry linked with elemental analysis), U.S. Pat. No. 7,135,296

(Elemental analysis of tagged biologically active materials), published patent application 2008/0046194, and Bandura et al Analytical Chemistry 2009 81 6813-6822 which are incorporated by reference for disclosure of those components.

In an alternative embodiment, prior to cleavage of the mass tag, individual particles may be deposited in a spatially separated manner on a substrate (e.g., a plate or wells of a microfluidic device). While on the substrate, the particles may be subjected to a cleavage stimulus, thereby releasing the mass tags from the particles. Leaving the particle on the substrate, a sample of the deposited material may be removed and subjected to elemental analysis. A cell having desirable properties can be retrieved from the substrate by obtaining coordinates for the cell, and they removing the cell from the substrate, e.g., by laser pulses or another suitable method. In certain emodiments, the cell may be deposited into culture medium after it is removed from the substrate.

### Kits

Also provided by the present disclosure are kits for practicing the method as described above. The subject kit contains reagents for performing the method described above and in certain embodiments may contain a plurality of labeled specific binding reagents, wherein each of the labeled specific binding reagent specifically binds a different target and each of the metal tags are distinguishable from one another by elemental analysis. The targets to which the specific binding reagents bind are on a cell surface. The kit may also contain a reference sample to which results obtained from a test sample may be compared.

In addition to above-mentioned components, the subject kit may further include instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate. In addition to above-mentioned components, the subject kit may include software to perform comparison of a collected hybridization signal with another.

### Utility

The above-described method and apparatus find use in a variety of protocols such as but not limited to sample phenotyping methods, cell sorting or isolation methods, and sample purification methods. The method and apparatus have numerous uses, several of which are described below.

Exemplary sample phenotyping methods employing the above-described method include, for example, antigen identification, disease diagnostics, and the like. In certain embodiments, one or more gene products can be identified on the cell surface or in the cytoplasm of cells using specific monoclonal antibodies cleavably linked to elemental tags. Cleavage and elemental analysis of the cleaved elemental tags may direct cell sorting as described above, thereby separating and collecting populations of cells bearing certain combinations of gene products.

In certain embodiments, the above-described method may be used to diagnose and help treat certain conditions by sensing certain molecules. Specific binding reagents may be employed that selectively bind to, for example, cancer antigens and identify cells bearing such antigens. Such antigen-bearing cells may then be isolated, and their genomes may be sequenced to provide information for genotype-specific therapeutic strategies.

Exemplary cell sorting or isolation methods employing the above-described method include, for example, isolation and collection of cells bearing certain markers or producing certain desirable molecules. In certain embodiments, the above-described method may be used to identify lineage specific stem cells. Specific binding reagents may be employed that selectively bind to certain reporter proteins expressed only in the desired cells. The desired cells may then be selectively collected as described above.

In certain embodiments, the above-described method may be used to isolate target cells having a certain ploidy. For example, a specific binding reagent targeting a particular gene of interest may be linked to one of two different elemental tags. A population of cells may then be subjected to a quantity of the specific binding reagent representing both tags. While haploid cells will bear at most one of the different tags, some diploid cells may bear both distinct tags. Using the above-described method to select only cells having two distinct elemental tags, diploid cells may be isolated from a population consisting of both haploid and diploid cells.

In certain embodiments, the above-described method may be used in cell counting applications such as cancer diagnosis or blood analysis. Cells extracted from a patient may be labeled by a specific binding reagent configured to bind to a cancer or blood cell marker. Cells that are labeled may be counted and compared to the number of cells that are not labeled, thereby obtaining at a ratio of labeled to non-labeled cells. Ratios outside of a certain range may indicate that a tumor is of a certain type of cancer or that a patient has abnormal levels of certain blood components.

In certain embodiments, the above-described method may be used to identify cells producing antibodies against a desired antigen. In such embodiments, an antigen of interest is the specific binding reagent. Cells producing antibodies that bind to the antigen of interest will bind the labeled antigen at higher concentrations than cells not producing such antibodies. Those cells producing antibodies of interest may be isolated as described above.

In certain embodiments, the above-described method may be used to isolate living cells from a population. For example, a population of cells may be subjected to stress conditions such that many of the cells are killed. The population may then be treated with a specific binding reagent configured such that cleavage of an elemental tag occurs via a process carried out only in living cells. Living cells will cleave the tag and thus produce a signal that will be absent in dead cells. The living cells may thereby be separated from dead cells and further analyzed.

Exemplary sample purification methods employing the above-described method include, for example, protein purification, nucleic acid purification, purification of multi-component subcellular complexes, and the like. In certain embodiments, the above-described method may be used in affinity-purification protocols. Specific binding reagents configured to selectively label target molecules may be employed. Using the above-described method, labeled target molecules may be separated from unlabeled contaminant molecules and collected for further purification or experimentation.

What is claimed is:

1. A method of sample analysis comprising:
a) labeling a particle using a labeled specific binding reagent that comprise a cleavably linked elemental tag to produce a labeled particle,
b) passing the labeled particle through a flow cell of a mass cytometer;
c) cleaving the elemental tag from the labeled particle;
d) performing elemental analysis of the cleaved elemental tag of c), without destroying said particle, to produce data;
e) matching data for said particle with said particle; and
f) collecting said particle.

2. The method of claim 1, wherein said particle is a cell.

3. The method of claim 2, wherein said specific binding reagent binds to an epitope on the surface of said cell.

4. The method of claim 2, wherein said specific binding reagent binds to an epitope present intracellularly in said cell.

5. The method of claim 1, wherein said elemental analysis comprises ionizing said metal tag using inductively coupled plasma to produce ions followed by mass spectrometry analysis of said ions.

6. The method of claim 1, wherein said binding reagent is an antibody.

7. The method of claim 1, wherein said elemental tag is an element having an atomic number of 21-90.

8. The method of claim 1, wherein said data contains identities and abundance of the transition metal of said elemental tag.

9. A labeled specific binding reagent comprising:
a specific binding reagent that specifically binds an analyte;
an elemental tag; and
a cleavable linker that joins said specific binding reagent to said elemental tag.

10. The labeled specific binding reagent of claim 9, wherein said specific binding reagent is an antibody.

11. The labeled specific binding reagent of claim 9, wherein said cleavable linker is cleavable by a chemical, physical or enzymatic stimulus.

12. The labeled specific binding reagent of claim 9, wherein said elemental tag is an element having an atomic number of 21-29, 39-47, 57-79 or 89.

13. A kit comprising plurality of labeled specific binding reagents of claim 9, wherein each of said labeled specific binding reagent specifically binds a different target and each of said metal tags are distinguishable from one another by elemental analysis.

14. The kit of claim 13, wherein the targets to which said specific binding reagents bind are on a cell surface.

15. A mass cytometer comprising:
a) a flow cell comprising:
i. an input for injecting labeled particles that are labeled with a specific binding reagents each comprising a binding region that is cleavably linked to an elemental tag into said flow cell in single file;
ii. means for administering a cleavage stimulus to said labeled particles to cleave the elemental tag from the labeled particle as they pass through said flow cell to produce cleaved metal tags and unlabeled particles;
iii. a diverter for separating said cleaved metal tags from said unlabeled particles prior to exit of the cleaved metal tags from the flow cell;
b) an inductively coupled plasma mass spectrometry system operably connected to the exit of the flow cell for elemental analysis of said cleaved metal tags to produce data;
c) a collector for collecting said cells after they have exited said flow cell; and
d) a register for matching data for each of said cells with a collected cell.

16. The mass cytometer of claim 15, where said means for administering provides a chemical, physical or enzymatic stimulus that cleaves the elemental tag from the labeled particles.
